(19) United States
(12) Patent Application Publication
(10) Pub. No.: US 2021/0393145 A1
(43) Pub. Date: Dec. 23, 2021

(54) MULTIPLEXING NON-INVASIVE BIOSENSING DEVICE FOR MONITORING PHYSIOLOGICAL RESPONSES TO OPIOIDS

(71) Applicant: MUSE Biomedical, LLC, Chapel Hill, NC (US)

(72) Inventors: Goutam Kumar Raju Gadiraju, Chapel Hill, NC (US); Jennifer Marie Jacober, Chapel Hill, NC (US); Karthik Sridhar Kaundinya, Chapel Hill, NC (US); Pradham Venkata Kameswara Tanikella, Chapel Hill, NC (US); Tasneem Essader, Chapel Hill, NC (US)

(73) Assignee: MUSE Biomedical, LLC

(21) Appl. No.: 17/353,952

(22) Filed: Jun. 22, 2021

Related U.S. Application Data
(60) Provisional application No. 63/042,226, filed on Jun. 22, 2020.

(51) Int. Cl. A61B 5/0205; A61B 5/00

(52) U.S. Cl. CPC A61B 5/0205; A61B 5/0022

(57) ABSTRACT

Provided herein is a non-invasive biosensing device and method for monitoring opioid response using the non-invasive biosensing device. The non-invasive biosensing device comprises a data collection and communication element. Biosensing extensions are included wherein at least one biosensing extension measures at least one biological function selected from the group consisting of respiratory rate, respiratory strength and duration of respiration. The data collection and communication element receives the measures of biological function and determines indexes based on the measures to form an opioid response index as a summation of the indexes and transmits the opioid response index to a receiver.

### CROSS-REFERENCE TO RELATED APPLICATIONS

This application claims priority to pending U.S. Provisional Patent Appl. No. 63/042,226 filed Jun. 22, 2020, which is incorporated herein by reference.

### FIELD OF THE INVENTION

This invention is related to medical devices and, more specifically, non-invasive biosensing devices capable of monitoring physiological responses to opioids. The invention is also specific to an improved method of detecting or predicting opioid overdose.

### BACKGROUND

In the current opioid epidemic, many patients who are prescribed such drugs develop tolerances and serious addictions during therapeutic use. These addictions have resulted in numerous overdose deaths. No method exists to measure and predict tolerance and addiction development and solutions also do not exist for rapid detection and alert of sudden overdose.

The United States is currently experiencing an opioid epidemic. The National Institute on Drug Abuse reports that over 2 million Americans abuse opioid drugs. The US Centers for Disease Control and Prevention reports approximately 130 deaths from opiate overdose occur each day. Furthermore, the National Institutes of Health reports that approximately 30% of patients will abuse their opioid prescriptions. The CDC reports that 168,158,611 prescriptions were issued in 2018. In total, the Society of Actuaries has estimated that the epidemic has cost the United States approximately 631 Billion USD from 2015-2018 reflecting immense financial consequences of opioid addiction and overdose.

To date, no treatment protocol adequately accounts for the effects of developing opioid addictions. Devices in the space of detecting opioids are often employed in punitive measures, such as detecting opioids in the blood or urine in a lab setting for use as evidence in criminal punishment, or for use in addressing addictions only after an overdose has occurred. No device exist that actively monitors a patient's response to opioid prescriptions throughout the duration of a pain management plan.

There have been previous efforts to solve this problem as represented in U.S. Pat. No. 10,390,699, which is incorporated herein by reference. The teachings therein fail to provide an adequate solution. The teachings therein utilize only a heart rate sensor, which is incapable of reliably detecting an opiate overdose, resulting in a low false positive rate.

In spite of the ongoing effort, the art still lacks a preventative method for predicting opioid addiction and tolerance suitable for allowing physicians, family members, and patients to assess how a patient is responding to a pain management plan. The present invention provides a much needed biosensor based solution suitable for mitigating an opioid crisis.

### SUMMARY OF THE INVENTION

The invention is related to a non-invasive biosensing device for monitoring the development of physiological tolerance and psychological addiction behavior during opioid use.

A particular feature of the invention is the ability to allow a patient to prevent addiction by making them aware of possible over-medicating, constituting a preventative, rather than reactive, solution to fighting the opioid epidemic.

These and other embodiments, as will be realized, are provided in a non-invasive biosensing device. The non-invasive biosensing device comprises a data collection and communication element. Biosensing extensions are included wherein at least one biosensing extension measures at least one biological function selected from the group consisting of respiratory rate, respiratory strength and duration of respiration. The data collection and communication element receives the measures of biological function and determines indexes based on the measures to form an opioid response index as a summation of the indexes and transmits the opioid response index to a receiver.

Yet another embodiment is provided in a method for monitoring opioid response in a patient. The method comprises:

providing the patient with a non-invasive biosensing device comprising;
a data collection and communication element; and
biosensing extensions wherein at least one biosensing extension measures at least one biological function selected from the group consisting of respiratory rate, respiratory strength and duration of respiration;
placing the non-invasive biosensing device in functional relationship with the patient; receiving measurements of the biological function and determining indexes based on the measures to form an opioid response index as a summation of the indexes; and
transmitting the opioid response index to a receiver.

### BRIEF DESCRIPTION OF FIGURES

FIG. 1 is a schematic representation of an embodiment of the invention.

FIG. 2 is a schematic representation of an embodiment of the invention.

FIG. 3 is a flow-chart representation of an embodiment of the invention.

### DESCRIPTION

Provided herein is a non-invasive biosensing device which is wearable or attachable, and method of using the non-invasive biosensing device, intended for use in the healthcare and biomedical devices fields. More specifically, the present invention multiplexes a series of biological signals and produces an Opioid Response Index (ORI) which is a score indicating that the overall effect of drugs, preferably opioids, on a patient. Furthermore, this invention predicts opioid tolerance and transmits processed data to physicians, patients, and the patients selected network, thereby allowing those parties to monitor and/or modify the patient's drug regimen to maximize treatment safety. If an overdose is detected, the device alerts the patient's selected network as well as emergency services.

The present invention provides a non-invasive biosensing device and method for monitoring the development of physiological tolerance and psychological addiction behavior thereby allowing a patient to prevent addiction by making the patient aware of possible over-medicating. This invention provides a preventative, rather than reactive, solution to fighting the opioid epidemic.

The invention will be described with reference to the figures forming an integral, but non-limiting, part of the specification. Throughout the figures similar elements will be numbered accordingly.

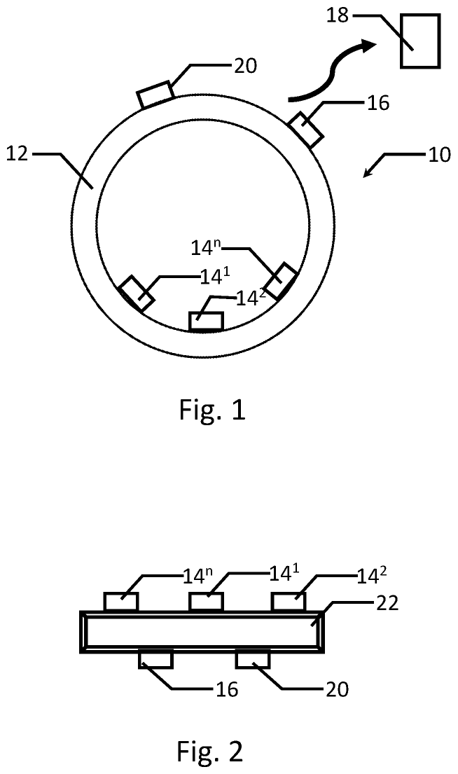

In one embodiment the non-invasive biosensing device is a wearable device which will be described with reference to FIG. 1. In FIG. 1, the wearable device, 10, is illustrated schematically. The wearable device comprises a band, 12, wherein the band circumnavigates a portion of the body, preferably the lower chest, in a position suitable for engagement of the various biosensing extensions, 14, with the body of the user in functional engagement capable of monitoring biological processes occurring in the body. A data collection and communication element, 16, interfaces with the biosensing extensions to determine an ORI based on a correlation of the data from the biosensing extensions and relays the ORI to a receiver, 18, which is remote from the wearable device. A power source, 20, such as a rechargeable battery provides power to the components of the non-invasive biosensing device.

In one embodiment the non-invasive biosensing device is an attachable device which will be described with reference to FIG. 2. In FIG. 2, the attachable device, 24, is illustrated schematically. The attachable device comprises a patch, 22, wherein the patch engages with a portion of the body, preferably the lower chest, in a position suitable for engagement of the various biosensing extensions, 14, with the body of the user in functional engagement capable of monitoring biological processes occurring in the body. A data collection and communication element, 16, interfaces with the biosensing extensions to determine an ORI based on a correlation of the data from the biosensing extensions and relays the ORI to a receiver, 18, which is remote from the wearable device. A power source, 20, such as a rechargeable battery provides power to the components of the non-invasive biosensing device.

The wearable device is preferably worn as a standalone accessory, taking the form of a single bodied wireless device preferably affixed to the lower chest.

The band is preferably adjustable and may be continuous and capable of being stretched to circumnavigate a portion of the body before being allowed to relax to functionally engage with the body. Alternatively, the band may have a portion which is reversibly engageable such that the band can be wrapped around the body prior to engageable portions being engaged to secure the band in functional engagement with the body. Clasps, buckles, hook and loop mechanisms such as Velco®, and the like are mentioned as suitable for demonstration of the invention.

The attachable device preferably comprises an adhesive or is secured to the body by a separate element such as a wrap, tape or the like.

The data collection and communication element comprises a wireless connectivity module configured for communication protocols such as Bluetooth Classic/Bluetooth Low Energy (BLE), Wi-Fi, Radio Frequency Communication (RF), or similar protocols.

For the purposes of this invention, a biosensing extension is an electrical or mechanical sensor that acquires and transmits data regarding biological processes occurring within/to the wearer's body, providing information on phenomena including but not limited to movement, oxygen saturation, or currents and electrical potentials within the body. Particularly preferred biosensing extensions include an Electrocardiogram (EKG) module, a pulse oximeter, an Electrodermal Activity (EDA) module, and an accelerometer. The biosensing extension comprises at least one biosensing extension that measures at least one biological function selected from the group consisting of respiratory rate, respiratory strength and duration of respiration. A diaphragmatic Electromyogram (EMG) module is included in a particularly preferred embodiment wherein the EMG is suitable for providing the respiratory rate, respiratory strength and duration of respiration as will be realized from the further description herein.

The data collection and communication element includes a microcontroller, microprocessor, or combined microcontroller and microprocessor control unit and wireless connectivity module which may be integral to a common component or separated components integral to the non-invasive biosensing device.

The data collection and communication element samples and analyzes the data from the biosensing extensions to provide an ORI which is correlated to a patient's Central Nervous System (CNS) depression resulting from usage of an opioid drug. The data collection and communication element transmits the ORI score through a Health Insurance Portability and Accountability Act (HIPAA) secure server to a preferably mobile application, where it may be relayed to a patient's designated care network, which may include their physician and family members. This score is quantified by an index referred to herein as the Opiate Response Index (ORI).

When the signals from the different sensors are recorded, they are combined and weighted to form the ORI. The EKG signal is processed to detect the heart rate. The EMG signal is processed with a fast-fourier transform and peak detector to extract respiration rate as well as the strength and duration of the diaphragm's contraction.

An accelerometer is used to determine when a patient's movement could confound the other signals, in which case the device stops processing signals to limit the amount of noise from movement artifacts. A pulse oximeter is preferably included to determine blood oxygenation, which is used in calculating the likelihood that a patient is experiencing an overdose.

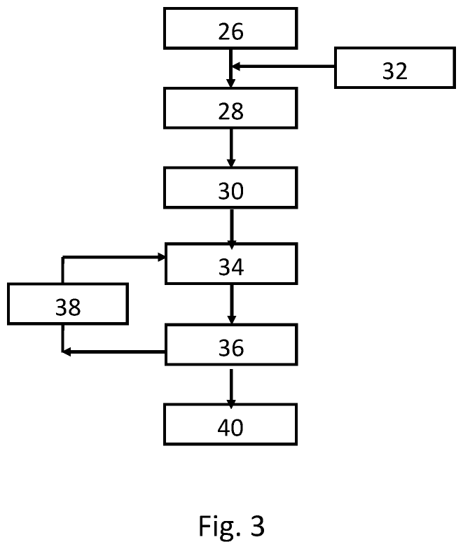

An embodiment of the invention will be described with reference to FIG. 3 wherein the method for determining the ORI is provided in a flow chart representation. In FIG. 3 the patient is provided with a non-invasive biosensing device in the form of a wearable device at 26. The base-line ORI is determined at 28. The base-line ORI is preferably before introduction of opioids at 30. In some instances, such as with trauma victims, the opioid may be introduced at 32 wherein the base-line ORI is not representative of an opioid-free measurement but is still useful for further analysis. Additional dosing continues at 34 followed by determination of usage ORI at 36. If the usage ORI is within preset parameters dosing and usage ORI determination continue at 38. If the usage ORI is not within preset parameters action is taken at 40 such as alerting the patient to the excursion from the preset parameters, contacting of predetermined family and medical practitioners, contact of EMS and the like.

In an embodiment of the invention it is preferable that for the first few days of starting opioid treatment, the device samples data every minute to determine how the patient's vitals trend at baseline and when they are either not taking the drug or taking low initial doses of the drug.

The ORI is defined as a scale, such as from 0 to 40, where 40 indicates normal baseline readings tailored to the patient and lower scores show progressive CNS depression. To calculate the ORI, each signal is rated on its own scale preferably with baseline being a higher index number than opioid response. For example, heart rate can be rated on a scale such as from 0 to 10, with 10 indicating a heart rate that is at or near baseline which is typically over 60 beats per minute (bpm) without limit thereto, 9 indicating a heart rate that is below baseline such as 50-60 bpm without limit thereto, 7 indicating a heart rate that is significantly below baseline such as 40-50 bpm without limit thereto, 4 indicating a very concerning heart rate such as 30-40 bpm with limit thereto, and a score of 0 if the heart rate is below a present lower limit such as 30 bpm without limit thereto. A progressive scale is preferably used so that signals which are farther from normal are weighted more heavily in the ORI score. A similar pattern is used for scoring other biological signals with the weighting factor for each being a selectable feature by the physician based on the physical characteristics of the patient. The sum of the measurements then forms the ORI and the physician can set predetermined levels of the ORI which are preset parameters such that within the preset parameters no action is necessary and outside of the preset parameters intervention is warranted.

In tandem with the non-invasive biosensing device a mobile application receives overall signal data from biosensing extensions as well as ORI scores over time. It is preferred that the mobile application communicate information about opioid levels effectively and efficiently to patients. ORI scores are preferably displayed on a graph for patients to see their overall data in simple format. The application also preferably allows a physician to program reminders for when a patient is safe to take another dosage of their prescribed drug. In such cases, a notification tells the user that they are approved to take the drug at the current time. The application preferably communicates both sensor data and ORI scores to the physician and designated family members, as approved by the patient. ORI data is also used to detect a possible narcotic overdose when the ORI score drops below a critical threshold as set by the physician or appropriate health care provider, at which point the non-invasive biosensing device signals the mobile application to alert emergency medical services (EMS), the physician, and designated family members or emergency contacts. ORI data is also used over long periods of time to determine if the patient is taking opioid drugs too often, or if the drugs no longer have the same effect on CNS depression at a given dose. All data transmission occurs through secured HIPAA compliant servers.

A key advantage of the non-invasive biosensing device is the ability to share patient critical information with the patient, their designated emergency contacts, and doctors. The family and doctors ensure that the patient is remaining safe with their opioid intake while following treatment protocols. At the same time the patient will be more informed about their health, which may make taking long-term pain medication less stressful. This can help change the way addiction is viewed and create a community to ensure healthy medicating habits.

### Examples

The invention has two primary pathways of use, depending on whether a patient first requires opioid medication treatment prior to using the non-invasive biosensing device or if they initiate use of the non-invasive biosensing device prior to initiation of opioid dosing. However, in both cases the baseline data specific to each patient is determined.

The first example of use pertains primarily to those patients who require pain medication as part of a treatment plan for an acute injury or surgical operation, for which they first receive care such as inpatient care in a hospital setting. For the inpatient setting, a physician may apply the non-invasive biosensing device to a patient to monitor how the patient's body responds to different types and doses of opioid medications. From the data produced by the non-invasive biosensing device, a physician may determine a threshold for the highest possible dose that may be applied to the patient without producing an overdose, allowing them to justify decisions to withhold or allow higher dosages of a drug to the patient. During this period of the time, the non-invasive biosensing device monitors and optionally reports the patient's unique physiological response to a given medication. Typically, patients will be prescribed opioid medications for pain management for a period of 1 week to 2 months. After being discharged, the non-invasive biosensing device is prescribed for continuous use throughout the period of opioid usage. During this period the non-invasive biosensing device fulfills a similar purpose to that described below for treatment beginning in the outpatient setting.

For patients undergoing long term pain management treatments including but not limited to while receiving chemotherapy or experiencing chronic pain, the non-invasive biosensing device is meant to be prescribed and worn for the entire period of opioid usage. For such patients, a treatment specialist instructs their patients to take an initial dose of a given medication based on clinical judgment and experience of the treatment specialist. In the following months, the non-invasive biosensing device collects data on how often the drug is taken and how the patient's body responds to the medication at its initially prescribed dose, and also aims to predict behavior indicative of psychological addiction and/or physiological tolerance behavior before an overdose has occurred or an addiction has developed. The patient's physician is able to set the timing interval of how often the patient is instructed to take their medication as well, and if this is done, the mobile application will provide the user with a reminder via notification to take their medication. The body's response to this dosing will then be recorded, confirming that the drug is taken at the expected interval. This data is shared amongst the patient and parties that the patient selects, including, for example, family members, loved ones, and physicians.

Processed ORI data is to be used by the physician to monitor that a patient is complying with their prescription and is also to be used in making decisions to alter the dosage of pain medication to reduce the likelihood of the patient developing an addiction or strong physiological tolerance to the drug. Both types of behavior would ultimately make the patient more susceptible to experiencing an overdose, as well as diminished quality of life and interpersonal interaction due to the negative psychological effects of addiction. Typically, a pain management specialist will check in with patients every 1-3 months in person to determine if the patient's current quality of life would be improved by altering the dosage or type of their opioid prescription. Previously, there has not been objective data that allows specialists to determine the safety of increased doses or to assess the degree of tolerance that a patient has developed. The non-invasive biosensing device provides the physician with objective data on both parameters in the form of trends in the ORI. Furthermore, by providing the patient's family with feedback on the safety of their patient's pain management treatment, this non-invasive biosensing device's usage offers peace of mind that a loved one is receiving closely monitored, safe, and adaptive care.

The non-invasive biosensing device also uses a continuous measurement of the ORI to detect a suspected overdose within 30 seconds of the overdose occurring. After the overdose is first detected, the non-invasive biosensing device has the capability to interact with a patient's mobile device to alert EMS and the patient's physician, as well as patient-designated parties, such as family members.

The invention has been described with reference to the preferred embodiments without limit thereto. One of skill in the art would realize additional embodiments and improvements which are not specifically stated but which are within the meets and bounds of the claims appended hereto.

Claimed is:

1. A non-invasive biosensing device comprising;
a data collection and communication element;
biosensing extensions wherein at least one biosensing extension of said biosensing extensions measures at least one biological function selected from the group consisting of respiratory rate, respiratory strength and duration of respiration; and
wherein said data collection and communication element receives said measures of said biological function and determines indexes based on said measures to form an opioid response index as a summation of said indexes and transmits said opioid response index to a receiver.

2. The non-invasive biosensing device of claim 1 wherein said data collection and communication element transmits said opioid response index to said receiver through a Health Insurance Portability and Accountability Act compliant server.

3. The non-invasive biosensing device of claim 1 wherein said data collection and communication element transmits said opioid response index to said receiver by a communication protocol selected from the group consisting of radiofrequency communication, Wi-Fi, or Bluetooth Classic/BLE protocols, or similar wireless communication protocols are used in the wireless communication module.

4. The non-invasive biosensing device of claim 1 further comprising at least one additional biosensing extension selected from the group consisting of a pulse oximeter, electrocardiogram module, an electrodermal activity module, an accelerometer and a diaphragmatic electromyogram module.

5. The non-invasive biosensing device of claim 1 wherein at least one of said respiratory rate, respiratory strength or duration of respiration is determined by a diaphragmatic electromyogram module.

6. The non-invasive biosensing device of claim 1 wherein said non-invasive biosensing device is wearable or attachable.

7. The non-invasive biosensing device of claim 1 further comprising a rechargeable battery.

8. A method for monitoring opioid response in a patient comprising:
providing said patient with a non-invasive biosensing device comprising;
a data collection and communication element; and
biosensing extensions wherein at least one biosensing extension of said biosensing extensions measures at least one biological function selected from the group consisting of respiratory rate, respiratory strength and duration of respiration;
placing said non-invasive biosensing device in functional relationship with said patient;
receiving measurements of said biological function and determining indexes based on said measures to form an opioid response index as a summation of said indexes; and
transmitting said opioid response index to a receiver.

9. The method for monitoring opioid response in a patient of claim 8 wherein said data collection and communication element transmits said opioid response index to said receiver through a Health Insurance Portability and Accountability Act compliant server.

10. The method for monitoring opioid response in a patient of claim 8 wherein said data collection and communication element transmits said opioid response index to said receiver by a communication protocol selected from the group consisting of radiofrequency communication; Wi-Fi, or Bluetooth Classic/BLE protocols, or similar wireless communication protocols are used in the wireless communication module.

11. The method for monitoring opioid response in a patient of claim 8 wherein said non-invasive biosensing device further comprises at least one additional biosensing extension selected from the group consisting of a pulse oximeter, electrocardiogram module, an electrodermal activity module, an accelerometer and a diaphragmatic electromyogram module.

12. The method for monitoring opioid response in a patient of claim 8 wherein at least one of said respiratory rate, respiratory strength and duration of respiration is determined by a diaphragmatic electromyogram module.

13. The method for monitoring opioid response in a patient of claim 8 wherein said placing said non-invasive biosensing device in functional relationship with said patient comprises said patient wearing or attaching said non-invasive biosensing device.

14. The method for monitoring opioid response in a patient of claim 8 wherein said non-invasive biosensing device further comprises a rechargeable battery.
